(19) United States
(12) Patent Application Publication
(10) Pub. No.: US 2010/0000862 A1
(43) Pub. Date: Jan. 7, 2010

(54) Integrated Blood Glucose Measurement Device

(75) Inventor: Anoop Rao, Cambridge, MA (US)

Correspondence Address: Larson & Anderson, LLC, P.O. BOX 4928, DILLON, CO, 80435, US

(73) Assignee: AgaMatrix, Inc., Salem, NH (US)

(21) Appl. No.: 12/498,227

(22) Filed: Jul. 6, 2009

Related U.S. Application Data
(60) Provisional application No. 61/078,740, filed on Jul. 7, 2008.

(51) Int. Cl. C12Q 1/54; G01N 27/26

(52) U.S. Cl. 204/403.02

(57) ABSTRACT

A portable combination for measuring a glucose concentration value in a sample has a portable glucose meter (GM) having a test strip port for receiving a disposable electrochemical test strip, means for calculating a glucose concentration value in a sample applied to a test strip received in the test strip port, and optionally a rechargeable GM battery. Next the combination has a portable rechargeable supplemental battery pack (SBP). The combination also has a web enabled portable device (WEPD) having a rechargeable WEPD battery and a wireless connection to the internet. The GM, the SBP, and the WEPD are electrically coupled to allow power transfer between the GM, the SBP, and the WEPD. The GM and the WEPD are communicatively coupled to allow for data transfer between the GM and the WEPD. The GM and SBP are detachable from and reattachable to the WEPD to form the portable combination. Lastly the combination has means for managing battery operations of the combination. These means are effective to cause the GM to draw operating power first from the SBP, second from the WEPD battery, and third from the GM battery, if the GM battery is present.

### CROSS REFERENCE TO RELATED APPLICATION

The present application claims priority from and is a non-provisional application of U.S. provisional application Ser. No. 61/078,740 filed on Jul. 7, 2008 which is herein incorporated for all purposes by reference.

### BACKGROUND

The use of web-enabled portable devices (WEPD) has allowed users of such devices the ability to access the web in many places. These WEPDs include hand-held devices such as cell phones (e.g. an IPHONE™, a BLACKBERRY™, or Palm Trio™, inter alia), personal data assistants, and web access assistants such as the ITOUCH™. These WEPDs allow users to connect to the world wide web (WWW) via wireless technology through wireless local area networks and/or through cell phone networks and towers, such as the EDGE™ and 3G™ networks, provided by AT&T. A user can download information as well as upload information to the WWW using these devices.

A user of these WEPDs may have some physical impairment or ailment which may require a periodic checks (e.g. once an hour or several times a day, week, and/or month) to determine whether a personal physiological characteristic is nearing or has reached a threshold level where physical danger is approaching or has approached. One example of such a user is a diabetic patient whom may be required to test for the concentration of glucose within their blood stream several times a day. This diabetic user typically carries on their person lancets for the acquisition of blood samples, a glucose meter (GM) or blood glucose meter (BGM), and disposable diagnostic test strips to which they apply the blood samples and insert into the blood glucose meter for analysis.

Much work has been done to make the analysis procedure for impaired patients as easy and pain free as possible. It would be extremely desirable to incorporate a diagnostic testing apparatus with a WEPD where possible to minimize the amount of equipment a patient is required to carry with them. However, due to processing power requirements and other power requirements of WEPDs (e.g. display power and wireless communication power requirements among others) the charge life of the self contained battery of the WEPD is often times short when the WEPDs are in operation and the addition of a separate device (e.g. a BGM) and the associated power requirements of such are often times not feasible. Furthermore, due to the memory and processor requirements of the software disposed in the WEPDs addition of advanced programs and equipment is not possible/preferable.

There is a need to integrate WEPDS with additional devices, such as medical diagnostic devices and to overcome the processing power and data storage requirements of programs offering complex calculations, such as diagnostic tests (e.g. those for complicated tests such as the concentration measurement of glucose in blood). The present invention provides for such improvements.

### SUMMARY OF INVENTION

The present invention provides a hand held portable combination of battery powered devices including a glucose meter (GM) and a web enabled portable device (WEPD), such as an IPHONE™ or an ITOUCH™ that solve the problems described above. The GM has means for calculating a glucose concentration value in a sample applied to a test strip received in a test strip port. The WEPD has a rechargeable WEPD battery and a wireless connection to the internet to transmit data about a test over the internet to a data receiving server. The portable combinations of the present invention include, inter alia, means for managing the battery power of the combined devices such that GM operational power is drawn from the individual component batteries in a specified order/protocol. In one embodiment, a portable combination for measuring a glucose concentration value in a sample comprises:

(A) a portable glucose meter (GM) having a test strip port for receiving a disposable electrochemical test strip, means for calculating a glucose concentration value in a sample applied to a test strip received in the test strip port, and optionally a rechargeable GM battery,
(B) a portable rechargeable supplemental battery pack (SBP),
(C) a web enabled portable device (WEPD) having a rechargeable WEPD battery and a wireless connection to the internet, wherein:

(I) the GM, the SBP, and the WEPD are electrically coupled to allow power transfer between the GM, the SBP, and the WEPD,

(II) the GM and the WEPD are communicatively coupled to allow for data transfer between the GM and the WEPD, and

(III) the GM and SBP are detachable from and reattachable to the WEPD to form the portable combination, and

(D) means for managing battery operations of the combination effective to cause the GM to draw operating power first from the SBP, second from the WEPD battery, and third from the GM battery, if the GM battery is present.
In another embodiment the present invention provides a portable combination for measuring a glucose concentration value in a sample comprising:
(A) a portable glucose meter (GM) having a test strip port for receiving a disposable electrochemical test strip, means for calculating a glucose concentration value in a sample applied to a test strip received in the test strip port, and optionally a rechargeable GM battery,
(B) a web enabled portable device (WEPD) having a rechargeable WEPD battery and a wireless connection to the internet, wherein:

(I) the GM and the WEPD are electrically coupled to allow power transfer between the GM and the WEPD,

(II) the GM and the WEPD are communicatively coupled to allow for data transfer between the GM and the WEPD, and

(III) the GM is detachable from and reattachable to the WEPD to form the portable combination,

(D) means for managing battery operations of the combination effective to cause the GM to draw operating power first from the WEPD battery and second from the GM battery, if the GM battery is present, and effective to cause the GM battery to be recharged first, if the GM battery is present, followed by the WEPD battery when the combination is connected to an external recharging power source, and
(E) calculated glucose value compilation means sufficient to: store the calculated glucose concentration value and previously calculated glucose concentration values; to analyze the calculated glucose concentration values; and to generate and communicate feedback and recommendations to the user regarding the analysis of the calculated glucose concentration values.
In yet another embodiment, the present invention provides a portable combination for measuring a glucose concentration value in a sample, the combination comprising:
(A) a portable glucose meter (GM) having a test strip port for receiving a disposable electrochemical test strip, means for calculating a glucose concentration value in a sample applied to a test strip received in the test strip port, and optionally a rechargeable GM battery,
(B) a portable rechargeable supplemental battery pack (SBP),
(C) a web enabled portable device (WEPD) having a rechargeable WEPD battery and a wireless connection to the internet, wherein:

(I) the GM and the SBP are electrically coupled to allow power transfer between the GM, and the SBP,

(II) the GM and the WEPD are communicatively coupled to allow for data transfer between the GM and the WEPD, and

(III) the GM and SBP are detachable from and reattachable to the WEPD to form the portable combination, and

(D) means for managing battery operations of the combination effective to cause the GM to draw operating power first from the SBP and then from the GM battery, if the GM battery is present, and effective to cause the GM battery to be recharged first, if the GM battery is present, followed by the SBP, when the combination is connected to an external recharging power source.

### BRIEF DESCRIPTION OF THE DRAWINGS

FIG. 1 is a top view of a WEPD connected to a SBP which is in turn connected to a GM.

FIG. 2A is a top view of an WEPD connected to a combined SBP/GM formed integral in the same housing.

FIG. 2B is a top view of an IPHONE™ connected to a combined SBP/GM formed integral in the same housing.

FIG. 2C is an exploded top view of an IPHONE™ about to be connected to a combined SBP/GM formed integral in the same housing.

FIG. 3A is a top view of an WEPD connected to a GM.

FIG. 3B is a top view of an IPHONE™ connected to a GM.

FIG. 4 is a top view of an IPHONE™ connected to a combined SBP/GM formed integral in the same housing.

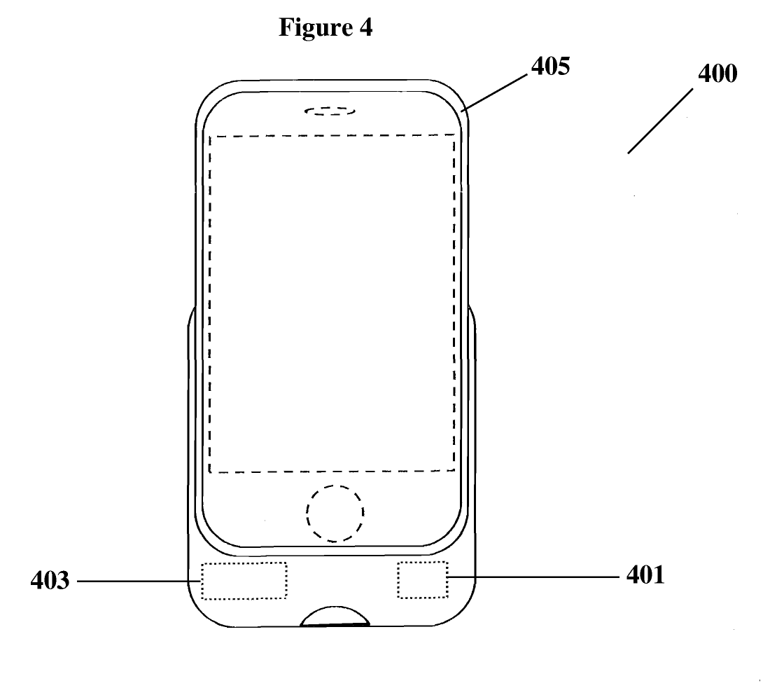

### DETAILED DESCRIPTION OF THE INVENTION

The use of a rechargeable battery powered WEPDs such as a web-enabled cellular telephone (e.g. a BlackBerry™, an IPHONE™, and/or a Palm Trio™) or web-enabled portable device (e.g. an ITOUCH™) in combination with a rechargeable battery powered diagnostic measurement device such as an glucose meter (GM) (e.g. a blood glucose meter (BGM)) is provided. Throughout the remaining text the diagnostic measurement device will be described within the context of an exemplary embodiment with the diagnostic measurement device is an analyte measurement device that measures the concentration of glucose within a sample (e.g. the diagnostic measurement device will be described in the context of a GM, more specifically a blood glucose meter (BGM)). Nothing within the following description however will limit the diagnostic device to a GM unless such context is so limiting, as other diagnostic devices are contemplated (e.g. blood pressure, body temperature, and other physiological characteristic monitors) and do not depart from the scope of this disclosure.

Power consumption by WEPDs as well as GMs with high computing power and/or rich displays accounts for fast battery drain of these devices. To solve this problem it has been found to use a supplemental battery pack/supply (SBP). These battery supplies (SBPs) are useful to retard the drain of the battery of these devices thereby extending the in-between-charge life. For example, the supplemental power supply as provided by Mophie, Inc. (www.mophie.com) which was acquired by mStation, Inc. (www.mstation.com) on about Sep. 4, 2007, has been found to be a preferable backup power supply of WEPDs produced by Apple, Inc. such as the IPHONE™ and the ITOUCH™.

The embodiments described herein permit a GM to have a smaller size compared to others that require a large attached power source capable of storing enough energy to perform many diagnostic tests, power large complex displays, and store large amounts of data. The present GM/WEPD combinations provided herein are portable and can operate independent of each other.

### DEFINITIONS

Reference throughout the specification to “one embodiment,” “another embodiment,” “an embodiment,” “some embodiments,” and so forth, means that a particular element (e.g., feature, structure, property, and/or characteristic) described in connection with the embodiment is included in at least one embodiment described herein, and may or may not be present in other embodiments. In addition, it is to be understood that the described element(s) may be combined in any suitable manner in the various embodiments.

Numerical values in the specification and claims of this application reflect average values for a composition. Furthermore, unless indicated to the contrary, the numerical values should be understood to include numerical values which are the same when reduced to the same number of significant figures and numerical values which differ from the stated value by less than the experimental error of conventional measurement technique of the type described in the present application to determine the value.

### First Embodiment

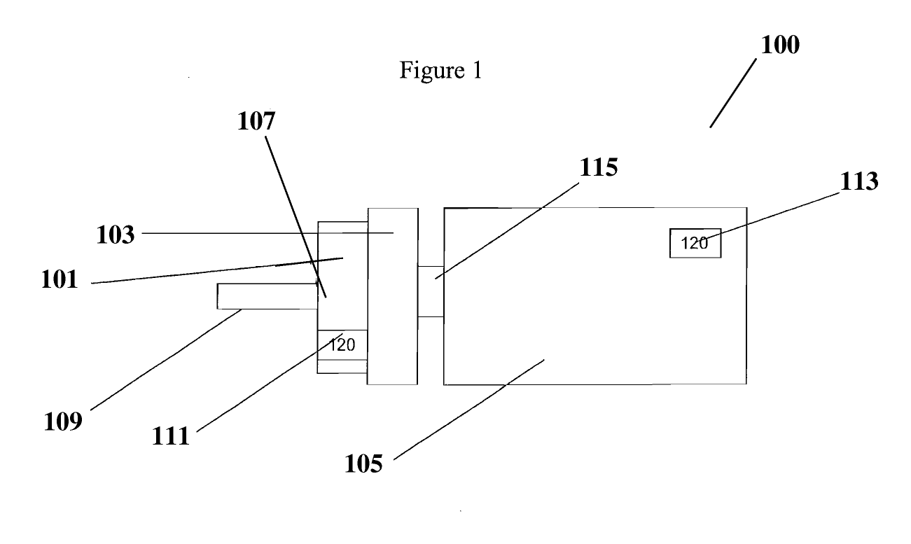

In a first embodiment of the present invention, as shown in FIG. 1, a portable combination 100 for measuring a glucose concentration value in a sample is provided. The portable combination comprises a portable GM 101, optionally having a rechargeable GM battery, connected to a portable rechargeable SBP 103 which is in turn connected to a portable WEPD 105 having a rechargeable WEPD battery and a wireless connection to the internet. The GM 101, the SBP 103, and the WEPD 105 are electrically coupled to allow power transfer between the GM 101, the SBP 103, and the WEPD 105 such that the GM can draw operating power from either or both the SBP 103 and the WEPD 105. The electrical connection can be either a wireless electrical transfer connection or more preferably a conventional wired electrical connection 115 (e.g. through the 30 pin connector of an IPHONE™ or ITOUCH™). The GM 101 and the WEPD 105 are communicatively coupled to allow for data transfer between the GM 101 and the WEPD 105. The communicative couple is either or both a wireless data connection (e.g. bluetooth, 802.11 wireless data transfer protocol, infrared, or some other wireless connection) and/or a wired connection from a data port on the GM 101 to a data port on the WEPD (105) (e.g. through the data pins or audio pins of the 30 pin connector of an IPHONE™ or ITOUCH™) (e.g.g. optionally through the SGP 103). The GM 101 and SBP 103 are detachable from and reattachable to the WEPD to form the portable combination 101. FIG. 1 shows electrical and communicative coupling between the SBP 103 (and hence the GM 101) to the WEPD 105 using a connector 115 (e.g. adapted to fit the 30 pin serial port of an IPHONE™ or ITOUCH™). Here the GM 101 and SBP 103 are detachable from the WEPD 105 and from each other. FIG. 1 also depicts the GM 101 and the WEPD 105 having optional displays 111, 113 disposed thereon which allow the user to see the results of a specific calculation and/or to receive feedback and/or recommendations based on the calculation or stored calculations. (e.g. a value of 120).

A strip port 107 located on the GM 101 allows diagnostic test strips (e.g. a disposable electrochemical test strip) to be inserted into the GM 101. The GM 101 contains means for calculating a glucose concentration value in a sample applied to a test strip 109 received in the test strip port 101. In FIG. 1 the calculation means include software contained in the GM specifically designed for measuring and calculation said value.

The combination 101 further comprises means for managing battery operations of the combination that are effective to cause the GM 101 to (I) draw operating power first from the SBP 103, second from the WEPD 105 battery, and third from the GM 101 battery, if the GM 101 battery is present. This ensures that power is depleted first from SBP 103 then from the WEPD 105 and lastly from the GM if it contains a battery. The ensures minimizing power draw and providing the most operational power remaining in the WEPD battery and lastly and most preferably in the GM battery, if present, to give a user the ability to perform a diagnostic test when other power sources have been depleted. The means for managing battery operations of the combination preferably are effective to cause the GM battery to be recharged first, if the GM battery is present, followed by the WEPD battery, and then the SBP, when the combination is connected to an external recharging power source such as to a wall socket, cigarette lighter, or wireless recharging power stations. Furthermore it is preferred that the combined device 100 is fully operational when connected to a recharging power source. In one embodiment the combination 100 allows the user to override the set battery charge dissipation protocol and input a specified charge dissipation protocol for a predetermined amount of tests (e.g. 1, 3, 5, or 10) before restoring the battery charge dissipation protocol in accordance with above. This allows a user to temporarily customize the battery charge dissipation protocol for a predetermined amount of time or test before it resets to the original programmed configuration outlined above.

In one embodiment a charging power source may be connected to the combinations described herein. The charging power source may be connected to any or all of the individual units in a flow through manner with a through power connection. For example, where the GM 101 is disposed distal from the WEPD 105 and where the external power source is connected between the GM 101 and the WEPD 105, the charging power source may be connected to the GM 101 such that the power flows through the GM 101 according to the above charging arrangement (e.g. where the GM 101 battery is charged first, the WEPD 105 is charged second, and the SBP 103 is charged third). Following a similar combination scheme where the external power source is disposed between the GM 101 and the WEPD 105 the charging power source may be attached to either the WEPD 105 or the SBP 103 following the same charging scheme as described. In another embodiment the GM 101 is disposed between the SBP 103 and the WEPD 105. In another embodiment the GM 101 is communicatively connected to the WEPD 105 through a direct wired connection (e.g. to an audio port) while it is electrically connected to the WEPD 105 through the SBP 103.

In a preferred embodiment, the SBP 103 supplies greater than 50% of the power required by the GM 101, more preferably greater than 75% of the power and most preferably 100% of the power required by the GM 101 prior to drawing power from the WEPD's 105 battery and the optional battery contained within the GM 101. In another preferred embodiment the means for managing battery operations of the combination are effective to discharge greater than 50%, for example greater than 75% and most preferably 100%, of the available power stored in the SBP 103 prior to drawing power from the WEPD 105 battery.

In preferred embodiments, where the GM 101 further comprises a GM 101 battery, the means for managing battery operations of the combination are effective to discharge greater than 50%, for example greater than 75% and most preferably 100%, of the available power stored in the SBP 103 prior to drawing power from the WEPD 105 battery. Where the WEPD 105 battery is tapped for GM 101 operating power it is preferred that the means for managing battery power are effective to discharge greater than 50%, for example greater than 75% and most preferably 100%, of the available power stored in the WEPD prior to drawing power from the GM battery (e.g. the SBP is depleted 50%, the WEPD batter is depleted 50%, and then power is allowed to be drawn from the GM battery, other combinations are contemplated such as 75% SBP, 75% WEPD and then GM battery draw or 100% SBP, 75% WEPD, and then GM battery draw, or 100% SBP, 100% WEPD, and then GM battery draw). This allows for the GM 101 battery to be the last power source drawn upon and thus has the benefit of allowing the GM battery to maintain a charge (and thus be used to calculate a glucose concentration in a sample) when the other devices have lost power or about to lose power.

When a user performs a test they will insert a diagnostic test strip 109 (e.g. an electrochemical diagnostic test strip have electrodes) into test strip port 107 of the GM 101. A sample is then introduced to the diagnostic test strip 109 and the GM 101 will perform the necessary calculations to determine a glucose concentration within the sample. The test results may then transmitted to the WEPD 105 of the combined device either automatically or in response to a predefined stimulus (e.g. depression of a button on the WEPD 101, the GM 101, or the SBP 103, or in response to a triggered condition such as a time parameter or glucose level alarm condition, inter alia). The WEPD 105 may then communicate the results to a data receiving server via the cellular telephone network or via a http protocol using a wireless local area network or by some other communication means to another network either automatically or in response to a predefined stimulus. The data receiving server may be located for example in the user's home, the user's workplace, a doctor's office or some other location remote from the combination.

In preferred embodiments the combination further comprises calculated glucose value compilation means. The calculated glucose value compilation means are not particularly limited other than they are effective to: store the calculated glucose concentration value and previously calculated glucose concentration values; analyze the calculated glucose concentration values; and generate and communicate feedback and recommendations to the user regarding the analysis of the calculated glucose concentration values. In one embodiment these means are a computer program stored in the GM 101, the WEPD 105, or a combination thereof. In a most preferred embodiment the means allow test data to be stored within/on disc space contained in the GM 101 in case of a power failure (or absence of WEPD 105 electrical/communication connectivity).

In a further preferred embodiment, the WEPD 105 is communicatively coupled to a remote data receiving server through its wireless connection to the internet to transfer calculated glucose concentration values from the WEPD 105 to the remote data receiving server and to transfer data from the receiving server to the WEPD 105. In this embodiment, the remote data receiving server comprises the calculated glucose value compilation means that are effective to store the calculated glucose concentration value and previously calculated glucose concentration values, to analyze the calculated glucose concentration values, and to generate and display recommendations to the user regarding the analysis of the calculated glucose concentration values on the WEPD 105 or GM 101.

In most preferred embodiments the calculated glucose value compilation means can compile test results received from the GM 101 and compile the results to generate feedback and/or recommendations to the user regarding the user previous test results, trends, averages (e.g. 1-day averages, 7-day averages, 30 day averages, among others), and action recommendations (e.g. testing recommendations, insulin dosage recommendations, doctor visit recommendations, among others). The calculation of an individual result occurs within the GM 101 and the compilation means may reside in the GM 101, the WEPD 105 or in the data receiving server. The feedback and/or recommendations can be provided to the user in the form of text, images, audio, and/or video among other forms of visual, audible, and/or tactile feedback generated or delivered to the WEPD 105, the GM 101, and/or to the SBP 103 (the GM 101 and the WEPD 105 being preferred). Feedback scenarios also include, inter alia: (A) The user's tests and a bad test result is returned, the WEPD or GM plays a video that warns user of complications of a high glucose; (B) after testing the compilation means provides indication of to test next convenient time or location (e.g. test at 6 PM, however at 6 PM, the meter senses that the user is accelerating and revises the testing schedule to a point in time where the meter does not sense acceleration) (e.g. g. test at 6 PM, however recognizing from a synced calendar on the WEPD that the user is busy in a meeting (or on a plane), inter alia, the compilation means provides a revised testing recommendation when the user is next available); and/or (C) reminds user to test at a certain time and recommend that the user carry “extra strips” to last through the length of the upcoming business trip (pulled by syncing with the calendar) or recalculating a testing schedule depending on number of strips available/used.

In other preferred embodiments, input from a variety of sensors (e.g. temperature sensors, accelerometers, altimeters, inter alia) can be incorporated into the combined devices 100 to interpret and/or provide for regional corrections of the electrochemical test performed by the GM 101. As is already known, GM 101 results can be affected by several environmental parameters such as temperature, altitude, pressure, humidity etc. These sensors may be incorporated in the GM 101, the SBP 103, the WEPD 105 or any combination thereof (most preferably in the WEPD 105). In a most preferred embodiment, the WEPD 105 will have global positioning ability using either satellite locating means and/or cell tower triangulation that pinpoints the combination's location (e.g. by longitude and latitude) and a queries database(s) (e.g. location dependent weather databases that provide information about temperature, humidity, and atmospheric pressure inter alia, and/or topographical databases that provide information about elevation, inter alia). The query results received can be used to modify calculated results (e.g. in the GM 101, in the WEPD 105, or in the data receiving server) or the query results can be used by the GM 101 as correction factors in the calculation.

Furthermore the global positioning means can be used to determine the location of the combination and the user and to set measured “units” to the customs of the country where the combination and user are located. For example, the global positioning means can be used to determine that the combination and user are located in the United States where units of measurement are typically based upon the United States customary system (pound, foot, and ° F.). This determination then could be used to program the combination to provide units of measurement in these units of measurement. A further example is that the global positioning means can be used to determine that the combination 100 and user are located in Europe where units of measurement are typically based upon the Metric system (gram, meter, and ° C.). This determination then could be used to program the combination 100 to provide units of measurement in Metric.

In further preferred embodiments the combination 100 further comprises means sufficient to detect strip insertion into the test strip port of the GM 101. Detection of strip insertion can be accomplished by the strip insertion closing a circuit or by shorting two contacts resulting in a change in the resistance of an already closed circuit. Insertion detection is described in U.S. Pat. Nos. 4,627,445, 4,714,874, 4,999,582, 5,108,564, 5,266,179, 5282,950, 5,320,732, 5,352,351, 5,438,271, 5,526,120, and 5,593,390, all of which are incorporated herein by reference for all purposes.

Upon detection of strip insertion the GM 101 preferably then queries whether the GM 101 is electrically connected to the WEPD 105 or the WEPD 105 and the SBP 103. The query results received then are communicated to the means for managing battery operations to control the combinations 100 battery usage. For example, if the GM 101 and SBP 103 are connected to the WEPD 105, then the GM's 101 required power for performing a diagnostic test is drawn from the external battery source first and then from the WEPD's 105 battery, second. If the GM 101 and SBP 103 is not electrically connected to the WEPD, then the GM 101 testing is achieved by power derived from the SBP 103 first and secondarily from a battery optionally contained within the GM 101 itself. The preference of powering the GM is by draining the external battery first, then the WEPD second, followed by the optional battery contained in the GM itself. This method is preferred as it ensures that the GM 101 battery gets discharged after the SBP 103 and the WEPD 105 battery. Where the GM 101 contains a self-contained battery, the GM 101 can perform the diagnostic test/calculation independent of the SBP 103 or the battery of the WEPD 105. The results can displayed on the GM 101, the SBP 103, the WEPD 105, or combinations thereof (e.g. on the GM 101 and the WEPD 105).

In most preferred embodiments, the combined device 100 is sized and weighted to be easily carried in one hand by a user. Further it is preferred that the SBP 103 and GM 101 are formed to make a substantially natural extension of the WEPD 105. The weighting of the combined device 100 is contemplated to be between 2 and 30 ounces and more preferably between 4 and 20, ounces for example between 8 and 15 ounces.

### Second Embodiment

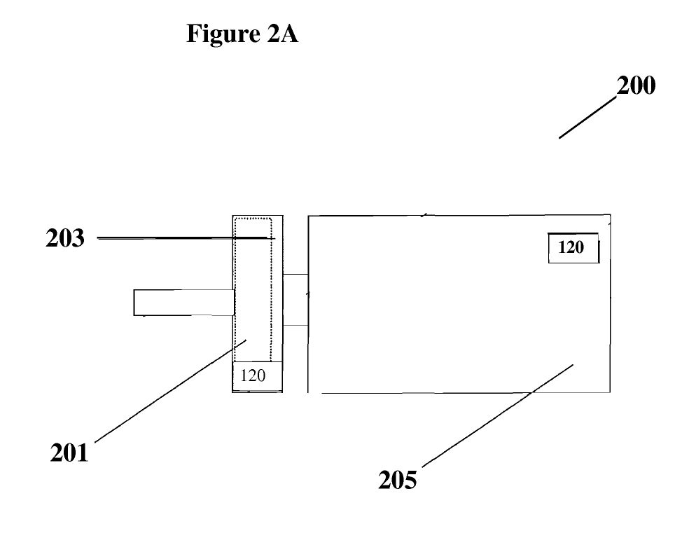

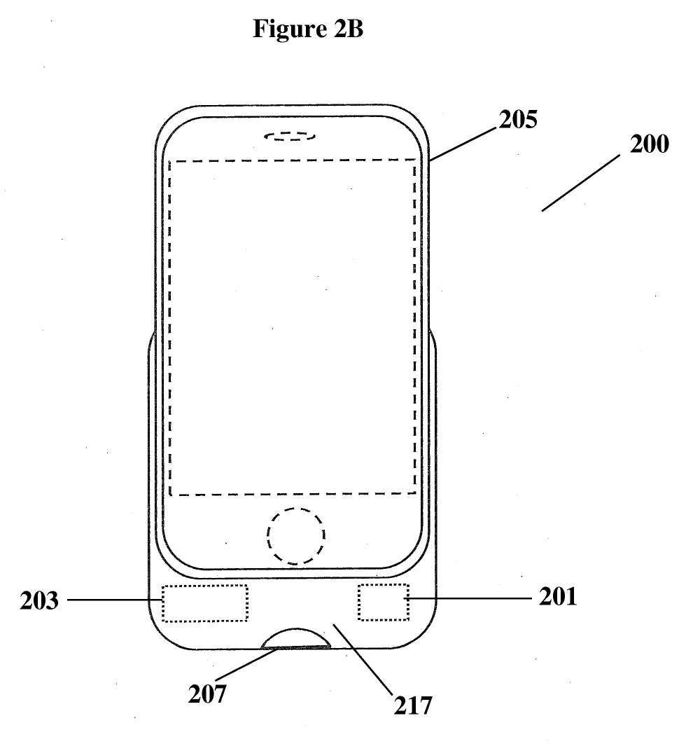

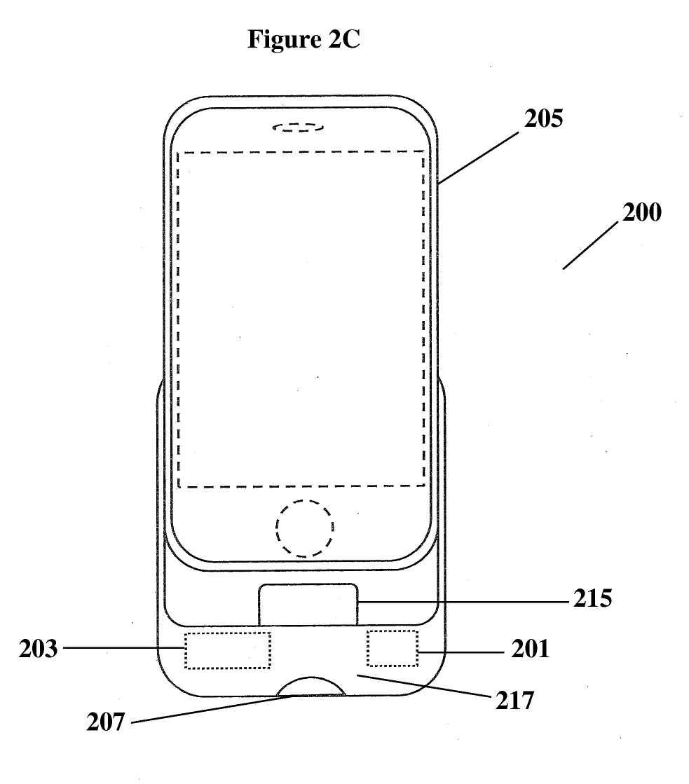

In the first preferred embodiment shown in FIG. 1 the GM 101, the SBP 103, and the WEPD 105 are formed in separate housings that are individually detachable from each other. As shown in FIG. 2A, a second preferred embodiment of the combination 200 of the present invention combines the GM 201 and SBP 203 into the same housing (e.g. they are formed integral in the same housing). In this embodiment, the SBP 203, the GM 201, and the WEPD 205 preferably function as described above with regard to FIG. 1, with the proviso that the GM 201 and the SBP 203 form a integrated and detachable unit that is separable from the WEPD 205. In this embodiment the GM again may or may not further comprise a GM battery.

FIGS. 2B and 2C show the combination 200 wherein the WEPD 205 is an IPHONE™ 205 and where the SBP 203 and the GM 201 are formed integral into an GM/SBP docking sled 217, with strip port 207, for attaching to the IPHONE™ 205. As shown in these figures the GM/SBP docking sled 217 when attached to the WEPD 205 forms a substantially natural extension of the WEPD 205 to allow a user to easily carry the combination 200 by hand, in a pocket, or bag. In one embodiment the combined device 200

FIG. 2C shows an exploded view of the combination 200 about to be formed. Here the docking sled 217 contains a connector 215 that inserts into the 30 pin connector/serial port of an IPHONE™ 205.

### Third Embodiment

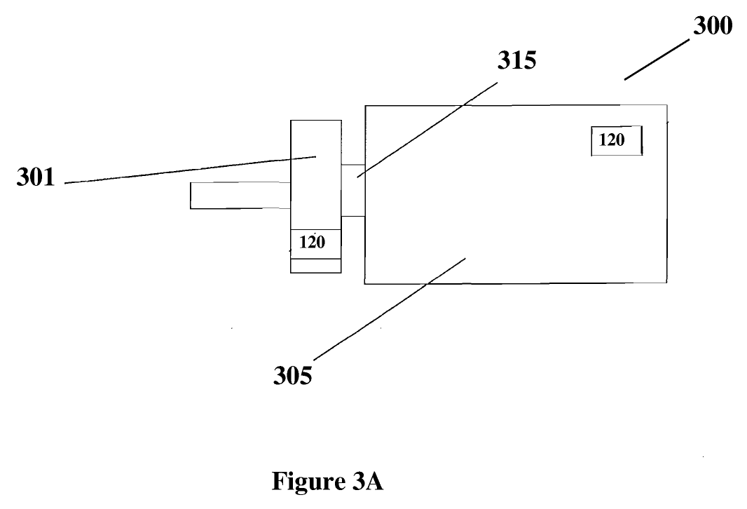

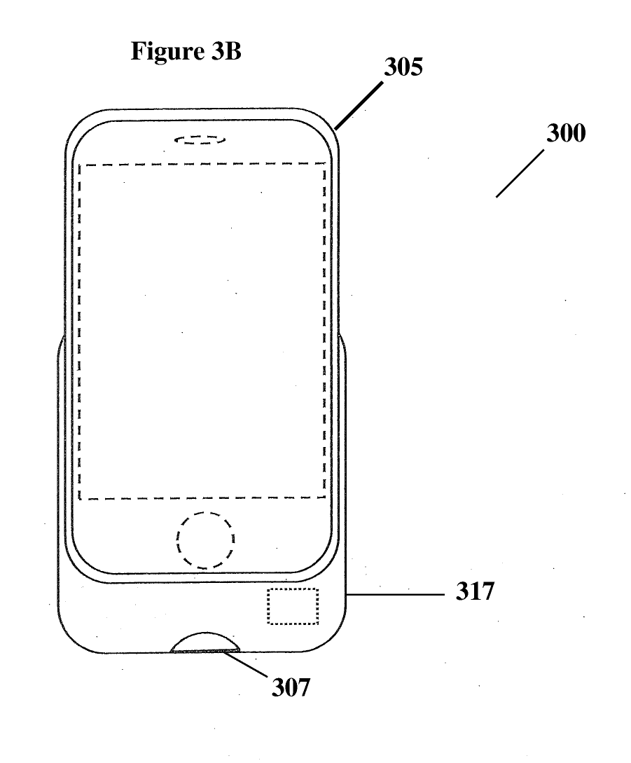

In a third embodiment, a combination as shown in FIG. 3A, the GM 301 communicates directly through a wired connection 315 with a serial port or some other port on the WEPD 305 or the GM 301 communicates wirelessly with the WEPD 305 as described above. In this embodiment no SBP is present and the GM 301 draws operational power required for its operation directly from the WEPD's 305 battery first, and then secondly from a battery optionally contained within the GM 301. FIG. 3B shows the combination 300 wherein the WEPD 305 is an IPHONE™ 305 and where the GM 301 is formed as a GM docking sled 317, with strip port 307, for attaching to the IPHONE™ 305. As shown in these figures the GM docking sled 317 when attached to the WEPD 305 forms a substantially natural extension of the WEPD 305 to allow a user to easily carry the combination 300 by hand, in a pocket, or bag.

The operations of the combined device 300 are as described with regard to the other embodiments described above with the exception that no SBP is present. Therefore the means for managing battery operations of the device 300 of this embodiment are different from the embodiments described above. In one embodiment, the GM 301 comprises a GM 301 battery and the means for managing battery operations of the combination are sufficient to discharge greater than 50%, for example greater than 75% (e.g. 100%), of the available power stored in the WEPD battery prior to drawing power from the GM battery.

### Fourth Embodiment

In fourth embodiment, the GM 401 is electrically connected to a SBP 403 but not electrically connected to the WEPD 405 to form the combination 400. In this Figure the GM 401 and the SBP 403 are shown integrated into the same housing although they may be formed in separate housings. The GM 401 and WEPD 405 are communicatively coupled either through a wireless connection, through a serial port data port (e.g. the data or audio pins), or some other port (e.g. an audio port), or a combination thereof to transfer data between the devices. The device 400 operates in a similar fashion to the embodiments described in either of embodiments one and/or two above with the proviso that no electrical transfer occurs between the WEPD 405 and the GM 401. Therefore the means for managing battery operations of the devices is different then these embodiments. Here the GM is allowed to draw from the SBP first and then second from the GM battery if present. The means for managing battery operations of the combined device 400 are sufficient to allow the GM 401 draw greater than 50%, for example greater than 75% (e.g. 100%), of the available power stored in the SBP 405 prior to drawing power from the GM battery, if it is present.

1. A portable combination for measuring a glucose concentration value in a sample, the combination comprising:
(A) a portable glucose meter (GM) having a test strip port for receiving a disposable electrochemical test strip, means for calculating a glucose concentration value in a sample applied to a test strip received in the test strip port, and optionally a rechargeable GM battery,
(B) a portable rechargeable supplemental battery pack (SBP),
(C) a web enabled portable device (WEPD) having a rechargeable WEPD battery and a wireless connection to the internet, wherein:
(I) the GM, the SBP, and the WEPD are electrically coupled to allow power transfer between the GM, the SBP, and the WEPD,
(II) the GM and the WEPD are communicatively coupled to allow for data transfer between the GM and the WEPD, and
(III) the GM and SBP are detachable from and reattachable to the WEPD to form the portable combination, and
(D) means for managing battery operations of the combination effective to cause the GM to draw operating power first from the SBP, second from the WEPD battery, and third from the GM battery, if the GM battery is present.

2. The combination of claim 1, further comprising calculated glucose value compilation means effective to: store the calculated glucose concentration value and previously calculated glucose concentration values; analyze the calculated glucose concentration values; and generate and communicate feedback and recommendations to the user regarding the analysis of the calculated glucose concentration values.

3. The combination of claim 2, wherein the calculated glucose concentration values are stored within the GM.

4. The combination of claim 1, wherein the WEPD is communicatively coupled to a remote data receiving server through its wireless connection to the internet to transfer calculated glucose concentration values from the WEPD to the remote data receiving server.

5. The combination of claim 4, wherein the remote data receiving server comprises calculated glucose value compilation means effective to store the calculated glucose concentration value and previously calculated glucose concentration values, to analyze the calculated glucose concentration values, and to generate and display recommendations to the user regarding the analysis of the calculated glucose concentration values on the WEPD or GM.

6. The combination of claim 1, wherein the GM, the SBP, and the WEPD are formed in separate housings that are detachable from each other and reattachable to each other to form the combination.

7. The combination of claim 1, wherein the GM and the SBP are formed in the same housing, wherein the WEPD is formed in a separate housing, and wherein the combined GM and SBP housing is detachable from the WEPD and reattachable to the WEPD to form the combination.

8. The combination of claim 7, wherein the GM does not have a GM battery.

9. The combination of claim 1, wherein the (D) means for managing battery operations of the combination are further effective to cause the GM battery to be recharged first, if the GM battery is present, followed by the WEPD battery, and then the SBP, when the combination is connected to an external recharging power source.

10. The combination of claim 1, wherein the (D) means for managing battery operations of the combination are effective to cause the GM to draw greater than 50% of its operating power from the SBP prior to drawing power from the WEPD battery.

11. The combination of claim 1, wherein the (D) means for managing battery operations of the combination are effective to cause the GM to draw greater than 75% of its operating power from the SBP prior to drawing power from the WEPD battery.

12. The combination of claim 1, wherein the (D) means for managing battery operations of the combination are effective to cause the GM to draw greater than 100% of its operating power from the SBP prior to drawing power from the WEPD battery.

13. The combination of claim 1, wherein the (D) means for managing battery operations of the combination are are effective to discharge greater than 50% of the available power stored in the SBP prior to drawing power from the WEPD battery.

14. The combination of claim 1, wherein the (D) means for managing battery operations of the combination are effective to discharge greater than 75% of the available power stored in the SBP prior to drawing power from the WEPD battery.

15. The combination of claim 1, wherein the (D) means for managing battery operations of the combination are effective to discharge 100% of the available power stored in the SBP prior to drawing power from the WEPD battery.

16. The combination of claim 1, wherein the GM further comprises a GM battery, wherein the (D) means for managing battery operations of the combination are effective to discharge greater than 50% of the available power stored in the SBP prior to drawing power from the WEPD battery, and are effective to discharge greater than 50% of the available power stored in the WEPD prior to drawing power from the GM battery.

17. The combination of claim 1, wherein the GM further comprises a GM battery, wherein the (D) means for managing battery operations of the combination are effective to discharge greater than 75% of the available power stored in the SBP prior to drawing power from the WEPD battery, and are effective to discharge greater than 75% of the available power stored in the WEPD prior to drawing power from the GM battery.

18. The combination of claim 1, wherein the GM further comprises a GM battery, wherein the (D) means for managing battery operations of the combination are effective to discharge greater than 100% of the available power stored in the SBP prior to drawing power from the WEPD battery, and are effective to discharge greater than 75% of the available power stored in the WEPD prior to drawing power from the GM battery.

19. The combination of claim 1, wherein the GM further comprises a GM battery, wherein the (D) means for managing battery operations of the combination are effective to discharge greater than 100% of the available power stored in the SBP prior to drawing power from the WEPD battery, and are effective to discharge greater than 100% of the available power stored in the WEPD prior to drawing power from the GM battery.

20. The combination of claim 1, wherein the WEPD is an IPHONE™ or an ITOUCH™ and wherein the GM is a blood glucose meter.

21. The combination of claim 1, further comprising means effective to detect strip insertion into the test strip port of the GM, upon detection of strip insertion to query whether the GM is electrically connected to the WEPD or the WEPD and the SBP, and communicating a query result to the (D) means for managing battery operations to control the combination battery usage.

22. The combination of claim 1, further comprising environmental sensor means for sensing and providing regional corrections for the electrochemical test performed by the GM.

23. The combination of claim 22, wherein the WEPD comprises said environmental sensor means.

24. The combination of claim 1, wherein the combination is sized and weighted to be easily carried in the hand of a user.

25. The combination of claim 24, wherein the GM and the SBP are designed to form a substantial extension of the WEPD.

26. A portable combination for measuring a glucose concentration value in a sample, the combination comprising:
(A) a portable glucose meter (GM) having a test strip port for receiving a disposable electrochemical test strip, means for calculating a glucose concentration value in a sample applied to a test strip received in the test strip port, and optionally a rechargeable GM battery,
(B) a web enabled portable device (WEPD) having a rechargeable WEPD battery and a wireless connection to the internet, wherein:
(I) the GM and the WEPD are electrically coupled to allow power transfer between the GM and the WEPD,
(II) the GM and the WEPD are communicatively coupled to allow for data transfer between the GM and the WEPD, and
(III) the GM is detachable from and reattachable to the WEPD to form the portable combination,
(D) means for managing battery operations of the combination effective to cause the GM to draw operating power first from the WEPD battery and second from the GM battery, if the GM battery is present, and to cause the GM battery to be recharged first, if the GM battery is present, followed by the WEPD battery when the combination is connected to an external recharging power source, and
(E) calculated glucose value compilation means effective to: store the calculated glucose concentration value and previously calculated glucose concentration values; analyze the calculated glucose concentration values; and generate and communicate feedback and recommendations to the user regarding the analysis of the calculated glucose concentration values.

27. The combination of claim 26, wherein the GM comprises a GM battery, wherein the (D) means for managing battery operations of the combination are effective to discharge greater than 50% of the available power stored in the WEPD prior to drawing power from the GM battery.

28. The combination of claim 26, wherein the WEPD is an IPHONE™ or an ITOUCH™ and wherein the GM is a blood glucose meter, wherein the WEPD further comprises environmental sensor means for sensing and providing regional corrections for the electrochemical test performed by the GM.

29. The combination of claim 26, further comprising means are effective to detect strip insertion into the test strip port of the GM, upon detection of strip insertion to query whether the GM is electrically connected to the WEPD or the WEPD and the SBP, and communicating a query result to the (D) means for managing battery operations to control the combination battery usage.

30. A portable combination for measuring a glucose concentration value in a sample, the combination comprising:
(A) a portable glucose meter (GM) having a test strip port for receiving a disposable electrochemical test strip, means for calculating a glucose concentration value in a sample applied to a test strip received in the test strip port, and optionally a rechargeable GM battery,
(B) a portable rechargeable supplemental battery pack (SBP),
(C) a web enabled portable device (WEPD) having a rechargeable WEPD battery and a wireless connection to the internet, wherein:
(I) the GM and the SBP are electrically coupled to allow power transfer between the GM, and the SBP,
(II) the GM and the WEPD are communicatively coupled to allow for data transfer between the GM and the WEPD, and
(III) the GM and SBP are detachable from and reattachable to the WEPD to form the portable combination, and
(D) means for managing battery operations of the combination effective to cause the GM to draw operating power first from the SBP and then from the GM battery, if the GM battery is present, and effective to cause the GM battery to be recharged first, if the GM battery is present, followed by the SBP, when the combination is connected to an external recharging power source.
